SEQUENCE LISTING (2 sequences)

SEQ ID NO: 1 - protein, 24 aa, Synthetic construct
  MOD_RES 11: note = phophorylated threonine
GSRSRTPSLPTPPTREPKKVAVVR

SEQ ID NO: 2 - protein, 24 aa, synthetic construct
GSRSRTPSLPTPPTREPKKVAVVR

(19) United States
(12) Patent Application Publication
(10) Pub. No.: US 2026/0202374 A1
(43) Pub. Date: Jul. 16, 2026

(54) METHOD OF MANUFACTURING BIOSENSOR FOR DETECTING BIOMARKER OF ALZHEIMER'S DISEASE AND BIOSENSOR MANUFACTURED THEREFROM

(71) Applicant: NOVASCOPE BIOCHIPS INC., Dover, DE (US)

(72) Inventors: CHANG-FU KUO, Zhubei City (TW); SIAN-HONG CIOU, Zhubei City (TW); AO-HO HSIEH, Zhubei City (TW); YU-XIU LIN, Zhubei City (TW); JHAO-LIANG SIE, Zhubei City (TW); MANI GOVINDASAMY, Zhubei City (TW); CHI-HSIEN HUANG, Zhubei City (TW)

(73) Assignee: NOVASCOPE BIOCHIPS INC., Dover, DE (US)

(21) Appl. No.: 19/133,727

(22) PCT Filed: Jan. 17, 2024
(86) PCT No.: PCT/US24/11715

§ 371 Date: May 29, 2025

Related U.S. Application Data
(60) Provisional application No. 63/439,596, filed on Jan. 18, 2023.

(51) Int. Cl. G01N 27/327; G01N 33/543; G01N 33/68

(52) U.S. Cl. CPC G01N 27/3276; G01N 27/3278; G01N 33/5438; G01N 33/6896; G01N 2333/46; G01N 2800/2821

(57) ABSTRACT

The present disclosure provides a method of manufacturing a biosensor for detecting a biomarker of Alzheimer's disease, comprising steps of depositing an aluminum oxide film on a Si substrate by an atomic layer deposition system to form an Al₂O₃/Si substrate; depositing electrical contacts Cr/Au on the Al₂O₃/Si substrate by a thermal evaporator to form a source, a drain and a planar gate on the Al₂O₃/Si substrate; providing a bilayer graphene on the Al₂O₃/Si substrate by thermal annealing under a vacuum environment; providing a bilayer graphene to a low-damage plasma treatment (LDPT) with a mixture of oxygen and hydrogen to form a graphene oxide/graphene (GO/G) layered composite on the Al₂O₃/Si substrate; and immobilizing an antibody on a surface of the GO/G layered composite through a reaction between amine groups of the antibody and carboxyl groups of GO of the GO/G layered composites, wherein the antibody is specific for p-tau217 protein.

### CROSS-REFERENCE TO RELATED APPLICATION

This application is a U.S. national stage application under 35 U.S.C. § 371 of International Application No. PCT/US24/11715, filed Jan. 17, 2024, which claims the benefit of U.S. Provisional Application No. 63/439,596, filed Jan. 18, 2023, the contents of which are incorporated herein by reference.

### REFERENCE TO AN ELECTRONIC SEQUENCE LISTING

The content of the electronic sequence listing (sequencelisting.xml; size: 3.04 kb; and date of creation: Jan. 12, 2024) is herein incorporated by reference in its entirety.

### BACKGROUND OF THE INVENTION

### 1. Field of the Invention

The present disclosure relates to a method of manufacturing a biosensor, and in particular to a method of manufacturing a biosensor for detecting a biomarker of Alzheimer's disease and a biosensor manufactured therefrom.

### 2. Description of the Related Art

Dementia is characterized by a loss in cognitive function beyond what might be expected from the usual consequences of biological aging. According to the World Health Organization report in 2021, there are currently more than 55 million people living with dementia worldwide; this number is projected to increase to more than 78 million in 2030 and 139 million in 2050 (Dementia, Newsroom, World Health Organization, 2022). It has become a serious threat to our society and health care system. Alzheimer's disease (AD) is the most common form of dementia and represents more than 60% of dementia cases. Clinical pathological evidence suggests that cognitive decline could herald clinical detection by more than 15 years (Amieya et al., 2014; Villemagne et al., 2013). Although AD progression is irreversible, effective treatments exist when AD is managed in its early clinical course. Therefore, early diagnosis and treatment have important clinical significance for the clinical management for AD.

Biomarkers are biochemical indicators used to judge disease risk. Among biomarkers, amyloid-β (Aβ) peptide accumulation in extracellular plaques and hyperphosphorylated tau (p-tau) protein accumulation in neurofibrillary tangles have been incorporated into the diagnostic framework for AD (Huang et al., 2009; Varesi et al., 2022; Teunissen et al., 2022). Aβ and p-tau can be visualized by positron emission tomography (PET) or measured quantitatively in the cerebrospinal fluid for AD diagnosis (Valotassiou et al., 2018; Brier et al., 2016; Palmqvist et al., 2015). However, these methods are invasive, bulky, expensive, and not widely available for all AD patients. Therefore, there is a strong demand to develop blood-based biomarkers for AD diagnosis because the collection of peripheral blood is simple, low cost and less invasive. Recent studies have shown that tau protein also enters the blood after its injection into the brain, and high p-tau levels in the blood indicate degeneration of neurons in the brain, which is a critical factor that ultimately leads to AD (Fiandaca et al., 2015; Banks et al., 2017; Chiu et al., 2013). Several studies with different assays and techniques demonstrated that p-tau protein isofornms, including p-tau181, p-tau217, and p-tau231, are highly specific for the detection of PET-confirmed Aβ and tau pathology across the clinical AD continuum (Suirez-Calvet et al., 2020; Bayoumy et al., 2021; Karikari et al., 2022; Leuzy et al., 2021) in phosphorylation, including electrolyte-insulator-semiconductor device (Bhalla et al., 2014; Bhalla et al., 2015), localized surface plasmon resonance (Bhalla et al., 2015) and electrochemical sensor (Formisano et al., 2015]. However, the sensitivity of p-tau protein detection is still very challenging due to the low concentration of p-tau protein in the peripheral blood, which is beyond the detection range of conventional enzyme-linked immunosorbent assays, and due to nonspecific proteins and various interferents when detecting it in blood samples (Derkus et al., 2016: Galasko et al., 2013; Lue et al., 2017; Yang et al., 2018; Hampel et al., 2018). Therefore, it is urgent to develop a point-of-care (POC) biosensor to detect the tau protein accurately in blood-based samples. Nanomaterial-based immunosensors have been reported to detect the p-tau protein. M. E. Schneider et al. prepared a carbon screen-printed electrode modified with platinum nanoparticles decorated with multiwall carbon nanotubes to develop an electrochemical-based immunosensor for the detection of p-tau181 (Schneider et al., 2022). To interact with the carboxyl group of the antibody for p-tau181 detection, the electrode was functionalized with amine groups using polyallylamine hydrochloride. Square-wave voltammetry was performed to detect p-tau181 in phosphate-buffered saline (PBS). The biosensor showed a limit of detection (LOD) of 0.24 pg/mL (~1.1 nM) with a linear range from 8.6 to 1100 μg/mL (~4.1 to 523.8 nM). With a 10-fold dilution in fetal bovine serum, the sensing performance in terms of sensitivity and LOD decreased to approximately half of that dilution in PBS. K. Kim et al. used the Langmuir-Blodgett technique to prepare densely aligned carbon nanotubes to develop a chemiresistive sensor array for multiple core AD biomarkers (Kim et al., 2020). The device demonstrated a LOD of 2.72 fM with a linear range from I fM to 100 nM for p-tau181 detection in plasma. H. T. N. Le and S. Cho developed an electrochemical biosensor based on an interdigitated wave-shaped electrode via an activated self-assembled monolayer to preserve a specific antibody for p-tau231 detection (Le et al., 2022). Electrochemical impedance spectroscopy was performed to detect p-tau231 in human serum. The biosensor displayed a LOD of 140 μg/ml (~66.7 nM) with a linear range of 100 μg/ml to 10 ng/ml (~47.6 to 476.2 nM). The precise detection of p-tau231 was also demonstrated in terms of the low dissociation constant between the antibody and p-tau231. L. M. T. Phan and S. Cho developed a colorimetric gold nanoparticle-based aptablot to detect p-tau231 in human serum albumin (Phan et al., 2022). The color intensity varying with p-tau231 concentration could be analyzed by the naked eye or by a digital camera with the support of ImageJ software. This approach resulted in a LOD of 4.71 μg/ml (~2.2 nM) with a linear range of 0.064 to 1000 ng/ml (~30.5 nM to 476.2 μM). S. Janelidze et al. reported that p-tau217 shows a stronger correlation with the tau PET tracer [18F]flortaucipir and that individuals with abnormally increased [18F]flortaucipir retention are more accurately identified, indicating that it is more useful than other biomarkers in diagnosing AD (Janelidze et al., 2020). Unfortunately, a POC biosensor for detecting p-tau217 has not yet been reported.

Geim and Novoselov disclosed monocrystalline graphitic films (Novoselov et al., 2004). Due to its many excellent properties, such as conductivity, mechanical strength, biocompatibility, and high surface area, graphene (G) has been widely used to develop various types of biosensors (Kanagavalli et al., 2021: Justino et al., 2017; Jangir et al., 2022) be a transducer in biosensors due to its high sensitivity toward the binding of biological analytes containing aromatic bases, such as DNA, RNA, and proteins. However, the affinity between G and biological analytes is nonspecific, making it unsuitable for detecting them alone. Therefore, the G surface must be functionalized to facilitate the binding of the biorecognition elements for the subsequent detection or capture of target analytes. Pyrene-based liners, such as pyrenebutanoic acid succinimidyl ester and pyrenebutyric acid, are commonly used to functionalize the G surface via z-z interactions (Nekrasov et al., 2022; Hinnemo et al., 2017). These wet chemical processes for G surface functionalization take several hours or even days, and the resulting functionalization occurs via noncovalent processes, which are less stable than their covalent counterparts.

In our previous study (Govindasamy et al., 2022), we developed an atomically layered composite of graphene oxide/graphene (GO/G) using chemical vapor deposition (CVD)-grown bilayer graphene (BG) and our developed low-damage plasma treatment (LDPT), which is an atomic layer oxidation process to develop a chemiresistive biosensor. CVD-grown BG turned into GO/G through LDPT, which could realize atomic layer oxidation with mixed hydrogen and oxygen gases. Only the top layer of BG was functionalized with oxidative groups serving as active sites for forming covalent bonds with a biorecognition element (e.g., the antibody in the present disclosure). Furthermore, the conductivity of the bottom G was almost unchanged after LDPT, and it could act as a transducer to respond to the attachment of the target analytes onto the top GO conjugated with the biorecognition element via π-π interactions between the GO and G layers.

The present disclosure provides a solution-gated field effect transistor (SGFET) featuring an atomically layered composite of graphene oxide/graphene (GO/G) to detect the p-tau217 biomarker.

### BRIEF SUMMARY OF THE INVENTION

It is an object of the present disclosure to provide a biosensor with high sensitivity for detecting a biomarker of Alzheimer's disease.

To achieve at least the above object, the method of manufacturing a biosensor for detecting a biomarker of Alzheimer's disease includes steps of depositing an aluminum oxide film on a Si substrate by an atomic layer deposition system to form an Al₂O₃/Si substrate; depositing electrical contacts Cr/Au on the Al₂O₃/Si substrate by a thermal evaporator to form a source, a drain and a planar gate on the Al₂O₃/Si substrate; providing a bilayer graphene on the Al₂O₃/Si substrate across the source and the drain by thermal annealing under a vacuum environment; providing a bilayer graphene to a low-damage plasma treatment (LDPT) with a mixture of oxygen and hydrogen to form a graphene oxide/graphene (GO/G) layered composite on the Al₂O₃/Si substrate; and immobilizing an antibody on a surface of the GO/G layered composite through a reaction between amine groups of the antibody and carboxyl groups of GO of the GO/G layered composites, wherein the antibody is specific for p-tau217 protein.

In an embodiment, the method further includes a step of dispensing an epoxy resin-type adhesive to define a sensing area.

In an embodiment, the sensing area has a size in a range from 5×5 to 10×10 mm².

In an embodiment, the step of immobilizing the antibody on the surface of the GO/G layered composite is performed by incubating the sensing area with a volume in a range from 20 to 100 μl aliquot of 100 μg/ml of the antibody at a temperature in a range from 4 to 37° C. for 1 to 24 hours.

It is another object of the present disclosure to provide a method for detecting a biomarker of Alzheimer's disease.

To achieve this object, the method for detecting a biomarker of Alzheimer's disease includes a step of detecting an amount of p-tau217 protein in a sample with a biosensor, wherein the biosensor includes a Si substrate; an aluminum oxide film disposed on the Si substrate by an atomic layer deposition system to form an Al₂O₃/Si substrate; electrical contacts Cr/Au disposed on the Al₂O₃/Si substrate by a thermal evaporator; a graphene oxide/graphene (GO/G) layered composite formed on the Al₂O₃/Si substrate by a low-damage plasma treatment (LDPT); and an antibody immobilized on a surface of the GO/G layered composite through a reaction between amine groups of the antibody and carboxyl groups of GO of the GO/G layered composites, wherein the antibody is specific for the p-tau217 protein.

In an embodiment, an epoxy resin-type adhesive is dispensed to define a sensing area of the biosensor.

In an embodiment, the sensing area has a size in a range from 5×5 to 10×10 m₂.

In an embodiment, the antibody is immobilized on the surface of the GO/G layered composite by incubating the sensing area with a volume in a range from 20 to 100 μl aliquot of 100 μg/ml of the antibody at a temperature in a range from 4 to 37° C. for 1 to 24 hours.

### BRIEF DESCRIPTION OF THE DRAWINGS

FIG. 1 shows the process showing the method of manufacturing the biosensor of the present disclosure.

FIG. 2 is a schematic view showing the solution-gated field effect transistors.

FIG. 3 shows schematic images of (a) the GFET-based biosensor featuring GO/G layered composite and (b) p-tau217 protein bound with antibody immobilized on GO/G, (c) sensing mechanism based on Dirac point shift, and (d) photos of the measurement setup and the solution-gated graphene transistor (SGGT).

FIG. 4 shows (a) transmittance of BG before and after LDPT, (b) Raman spectra of BG before and after LDPT, (c) XPS spectra of BG before and after LDPT, (d) contact angles of BG before and after LDPT, (e) electrical resistances of SLG and BG before and after LDPT, and (f) TEM image and interlayer spacing of the atomically layered composite of GO/G.

FIG. 5(a) is a graph showing transfer curves of SGFETs comprising the BG and the GO/G atomically layered composite, and FIG. 5(b) is a graph showing output characteristics of the GO/G atomically layered composite.

FIG. 6(a) is a graph showing transfer curves of the antibody-modified GO/G-based SGFET, FIG. 6(b) is a graph showing VCNP with respect to the incubation time, and FIG. 6(c) shows a fluorescence microscopy image of the GO/G sample after exposure to the amine- and FAM-modified antibody (left) and the corresponding optical image (right).

FIG. 7(a) is a graph showing transfer curves of the antibody-modified GO/G-based SGFET, FIG. 7(b) is a graph showing ΔV_{CNP} with respect to the p-tau217 protein concentration in PBS, FIG. 7(c) is a graph showing carrier concentration with respect to p-tau217 protein concentration, and FIG. 7(d) is a graph showing mobility with respect to p-tau217 protein concentration, as obtained through Hall measurement.

FIG. 8(a) is a graph showing transfer curves of the antibody-modified GO/G-based SGFET, and FIG. 8(b) is a graph showing ΔV_{CNP} with respect to the p-tau217 protein concentration in HSA.

FIG. 9(a) is a graph showing transfer curves of the antibody-modified GO/G-based SGFET, and FIG. 9(b) is a graph showing V_{CNP} and variation recorded after different periods of storage.

### DETAILED DESCRIPTION OF THE INVENTION

To facilitate understanding of the object, characteristics and effects of this present disclosure, embodiments together with the attached drawings for the detailed description of the present disclosure are provided.

### Materials and Instrumentation

In the present disclosure, p-tau217 protein and its antibody, which was enriched with amine groups at the C-terminus, were obtained from Chang Gung Memorial Hospital, Taiwan. The details of protein preparation and colone screening and qualification check are described as follows.

Peptide synthesis and protein preparation: the purity of p-tau-217 peptide, GSRSRTPSLPTPPTREPKKVAVVR (SEQ ID NO: 1), and control peptide, GSRSRTPSLPTPPTREPKKVAVVR (SEQ ID NO: 2), was >95%, wherein the 11^{th} amino acid of SEQ ID NO: 1 is phosphorylated threonine, and the 11^{th} amino acid of SEQ ID NO: 2 is threonine without being phosphorylated, as per the manufacturers' guarantee (BIOTOOLS Co., Ltd, TW). The peptide was prepared and stored according to the manufacturer's recommendations (20 μg/μl). Tau (MAPT) protein was purchased from Origene (NM_005910, USA) and phosphorylated by glycogen synthase kinase 3 beta (GSK-3β, Sino Biological, China). Inactivated viral lysates, influenza A/B (Flu A/B), human parainfluenza virus, adenovirus, respiratory syncytial virus (RSV), and severe acute respiratory syndrome coronavirus (SARS) were purchased (ZeptoMatrix, USA) and used following manufacturers' instruction.

Immunization: 6-8 weeks-old Female BALB/c mice were received an intraperitoneal injection with 100 μg p-tau-217, emulsified with complete Freund's adjuvant (Sigma-Aldrich, USA) respectively. Boosting was performed with 100 μg peptide in incomplete Freund's adjuvant (Sigma-Aldrich, USA) on day 14, 28, and 42. Before sacrifice, Antibody response was provoked by injection with 50 μg emulsified with IFA twice at three-days intervals before sacrifice.

Hybridoma preparation and antibody purification: for generation of monoclonal antibody against p-tau-217 peptide, BALB/c mice were inoculated with synthetic p-tau-217 peptide for three times every two weeks. Boost was conducted twice at three-days intervals before sacrifice. Mice spleens were collected immediately and fused with myeloma cell for hybridoma preparation and semi-solid selection subsequently (ClonaCell Hybridoma kit, STEMCELL technology, USA). Hybridoma colonies were propagated in 96 well microtiter plate (Corning, USA) till cells at confluency and their supernatants were harvested to examine antibody response to p-tau-217 peptide, full length of p-tau protein or control peptide by ELISA test. The high affinity of antibodies to p-tau-217 peptide or/and p-Tau protein was selected for protein G Sepharose resin (Cytiva, USA). These purified monoclonal antibodies were dialysis with PBS buffer to remove glycine and concentrated with Amicon Ultra-15 centrifugal filters units (10 kDa, Merck Millipore, USA). Monoclonal antibodies were stored at −80° C. for following experiment.

Enzyme-linked immunosorbent assay (ELISA): 100 ng/well of p-tau protein, p-tau-217 peptide or tau-217 in coating buffer (150 mM Na2CO3, 150 mM NaHCO3, pH 9.6) was coated to a 96-well microtiter plate at 4° C. overnight. For cross-reactivity test, 1 μg/well inactivated viral lysates were used for test. After blocking with 1% bovine serum albumin, 100 μL cell supernatant or lug purified monoclonal antibodies were added and incubated at room temperature (RT) for 1 hours. At the end of the incubation, the microtiter plate was washed four times with TBST (TBS with 0.05% Tween 20) and bound antibody was detected by horseradish peroxidase (HRP)-conjugated anti-mouse IgG Fc region at a dilution of 1:2000 (Jackson ImmunoResearch Laboratories, USA) at room temperature for 1 hour. After washing, 3, 3′, 5, 5′-tetramethylbenzidine (TMB, Sigma Aldrich, USA) was used as the substrate and horseradish peroxidase (HRP) activity was read at 450 nm with a micro-ELISA reader (EZ read 400).

Modification of monoclonal antibody: the amine-reactive esters of carboxylate groups on monoclonal antibody were prepared by reactive amination kit following manufactures' recommendation (G-BIOSCIENCES, USA). Briefly, 1 mg/mL purified monoclonal antibody was dissolved in 1× optimizer buffer. EDC and Sulfo-NHS were added to give the final concentration of 2 mM and 5 mM with 15 minutes incubation at room temperature. Then, β-mercaptoethanol (Sigma Aldrich, USA) to a final concentration of 20 mM was added into antibody solution and incubated for 10 minutes at room temperature to inactivate EDC. The free EDC, NHS and EDC-byproducts were removed by desalting column and antibody solution, adjusted by PBS (pH 7), was concentrated by 10 kDa cutoff centrifugal filters units. The amine reactive site was blocked by hydroxylamine (Sigma Aldrich, USA) to a final concentration 10 mM for 5 minutes at room temperature. Buffer exchange was carried out with PBS (pH 7) by 10 kDa cutoff centrifugal filters. Antibody solution was aliquoted into 100 μL/vial (0.25-0.5 μg/μL) and stored at −80° C.

For preparation of monoclonal antibody (mAb) to p-tau 217, the immunization of BALB/c mice with p-tau 217 peptide was carried out to induce antibody against p-tau 217. At the end of immunization, mice spleen was harvested for hybridoma fusion with myeloid cells to generate hybridoma clones that producing anti-p-tau 217 mAb. At the preliminary selection, more than five hundreds of hybridoma clones were selected to examine the antibody reactivity to p-tau 217 peptide and p-tau protein. The top 20 clones present with higher reactivity to p-tau 217 and p-tau were listed in Table 1. Table I shows affinity test result of monoclonal antibody to p-tau protein and p-tau 217 peptide, in which O.D. is the abbreviation of optical density.

TABLE 1

| P-tau | | P-Tau 217 | |
|---|---|---|---|
| Clone No. | O.D.₄₅₀ | Clone No. | O.D.₄₅₀ |
| 118 | 1.213 | 118 | 1.457 |
| 49 | 0.99717 | 3 | 1.322 |
| 3 | 0.883 | 123 | 1.314 |
| 140 | 0.862 | 178 | 1.281 |
| 167 | 0.797 | 103 | 1.217 |
| 170 | 0.793 | 49 | 1.210 |
| 221 | 0.783 | 83 | 1.210 |
| 125 | 0.775 | 130 | 1.161 |
| 83 | 0.775 | 78 | 1.158 |
| 203 | 0.755 | 72 | 1.154 |
| 177 | 0.752 | 170 | 1.141 |
| 127 | 0.744 | 35 | 1.124 |
| 72 | 0.737 | 236 | 1.123 |
| 232 | 0.731 | 127 | 1.105 |
| 123 | 0.73 | 17 | 1.091 |
| 71 | 0.722 | 140 | 1.049 |
| 93 | 0.711 | 211 | 1.040 |
| 51 | 0.704 | 69 | 1.036 |
| 103 | 0.698 | 92 | 1.023 |
| 214 | 0.688 | 43 | 1.005 |

Ten clones of mAb (No. 3, No. 49, No. 72, No. 83, No. 103, No. 118, No. 123, No. 127, No. 140, and No. 170) were picked up to perform the limit of detection using ELISA test (Table 2). Table 2 shows the detection limit of monoclonal antibodies to p-tau 217 peptide.

TABLE 2

| | Clone No. | | | | | | | | | |
|---|---|---|---|---|---|---|---|---|---|---|
| pg/mL | 3 | 49 | 72 | 83 | 103 | 118 | 123 | 127 | 140 | 170 |
| 250 | 0.135 | 0.31 | 1.398 | 0.425 | 0.685 | 0.217 | 1.241 | 0.962 | 0.147 | 0.108 |
| 125 | 0.144 | 0.196 | 0.904 | 0.33 | 0.137 | 0.126 | 0.532 | 0.592 | 0.148 | 0.114 |
| 62.50 | 0.152 | 0.118 | 0.434 | 0.296 | 0.197 | 0.132 | 0.399 | 0.138 | 0.143 | 0.104 |
| 31.25 | 0.123 | 0.125 | 0.145 | 0.13 | 0.129 | 0.124 | 0.126 | 0.122 | 0.114 | 0.113 |
| 15.62 | 0.131 | 0.113 | 0.125 | 0.107 | 0.173 | 0.131 | 0.113 | 0.114 | 0.112 | 0.113 |
| 7.81 | 0.107 | 0.101 | 0.108 | 0.104 | 0.103 | 0.104 | 0.109 | 0.117 | 0.101 | 0.101 |
| 3.91 | 0.116 | 0.105 | 0.119 | 0.051 | 0.114 | 0.122 | 0.114 | 0.111 | 0.107 | 0.103 |
| 1.95 | 0.114 | 0.113 | 0.124 | 0.163 | 0.104 | 0.121 | 0.144 | 0.27 | 0.113 | 0.104 |

To further rile out the possibility of antibody cross-reacting to other antigens, two clones (No. 72 and No. 123) of mAb with higher affinity to p-tau 217 were examined for cross-reactivity with other viral antigens, e.g., influenza A/B, human parainfluenza virus, adenovirus, respiratory syncytial virus (RSV), and severe acute respiratory syndrome coronavirus (SARS). Also, p-tau 217 and tau 217 peptide were used as positive and negative controls (Table 3). Table 3 shows the cross-reactivity test result of monoclonal antibody to viral lysates, in which Flu A/B stands for influenza A/B, para-Flu stands for human parainfluenza virus, RSV stands for Respiratory syncytial virus, SARS stands for severe acute respiratory syndrome coronavirus, and control represents tau-217 peptide. The clone No. 72 with higher antibody affinity to p-tau-217 and p-tau, and few cross-reactivity with other antigens was used in this study.

TABLE 3

| Clone | Flu | Flu | Para- | | | | p-tau | |
|---|---|---|---|---|---|---|---|---|
| No. | A | B | Flu | Adenovirus | RSV | SARS | 217 | control |
| 123 | 0.133 | 0.552 | 0.072 | 0.074 | 0.068 | 0.045 | 1.775 | 0.065 |
| 72 | 0.05 | 0.101 | 0.191 | 0.094 | 0.109 | 0.045 | 1.894 | 0.074 |

1× phosphate buffered saline (PBS; containing 137 mM NaCl, 2.7 mM KCl, 8.1 mM Na₂HPO₄, and 1.47 mM KH₂PO₄) with pH 7.4 and HSA were purchased from Sigma-Aldrich Corporation, US. All other chemicals were of analytical grade and used as received. Deionized water was obtained from a Millipore water purification system (18.2 MQ resistivity, Milli-Q Direct 8). A 25-μm copper foil for bilayer graphene (BG) preparation was purchased from Alfa Aesar (Thermo Fisher Scientific). BG was grown on copper foil using CVD with a 3-inch diameter tubular quartz furnace. The details of the growth procedures and the subsequent transfer to a target substrate are described in Govindasamy et al., 2022.

Optical and structural characterizations of the G samples were carried out using ultraviolet-visible (UV-Vis) spectroscopy (V-650, JASCO Corp., Japan) and Raman spectroscopy (HORIBA iHR-550 equipped with a 532 nm laser, Japan). The chemical compositions were investigated using X-ray photoelectron spectroscopy (XPS; PHI 5000 VersaProbe III, ULVAC, Japan) with a monochromated Al Kα source. The layered composite was observed by a transmission electron microscope (TEM, JEOL, JSM-2100, Japan). Water contact angle (WCA) measurements were conducted to examine the hydrophilicities using a PSC-100B instrument (Pentad Scientific, Taiwan). The electrical properties of the SGFET were measured with a semiconductor parameter analyzer (B1500A, Agilent Tech., USA) in a homemade probe station. The source-drain current (I_{sd}) versus gate voltage (V_{g}) transfer curves were measured under a V_{g} of −0.4 to 1.2 V with an interval of 0.02 V and source-drain bias (V_{sd}) of 0.5 V. A Hall effect measurement system (AHM-800B, Agilent Tech., USA) was used to determine the carrier concentrations and mobilities of the devices subjected to different concentrations of p-tau217 solution. All properties of at least five samples were measured; averages and standard deviations are reported herein.

Method of Manufacturing Biosensor and p-Tau217 Detection Protocol

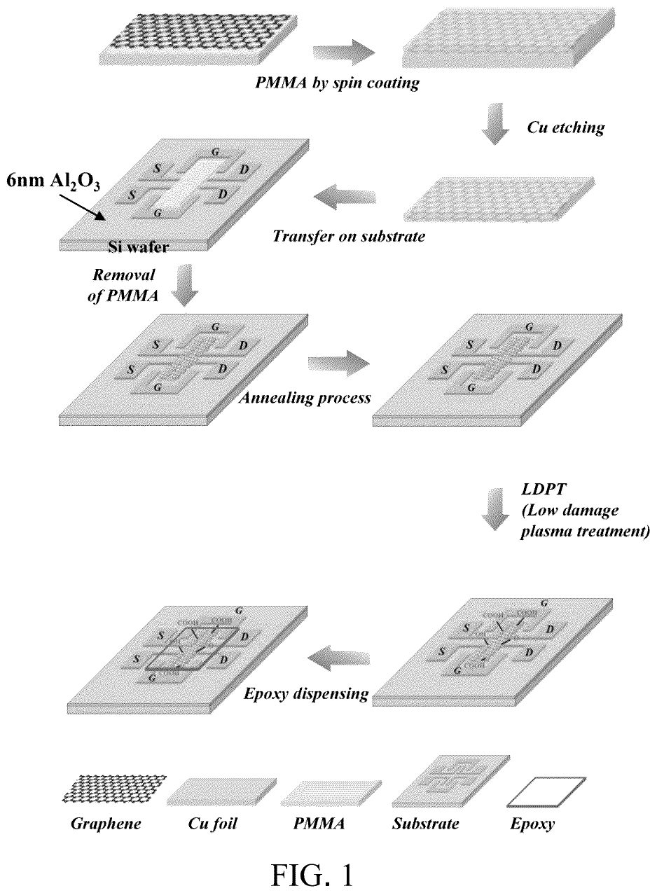

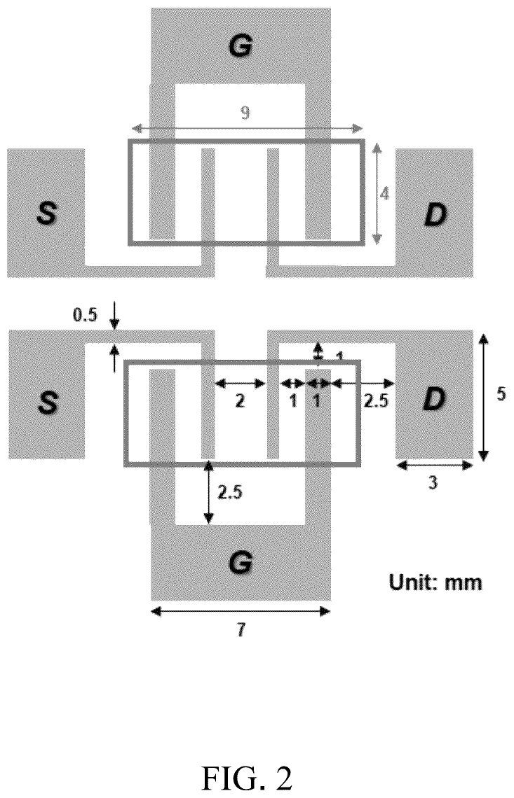

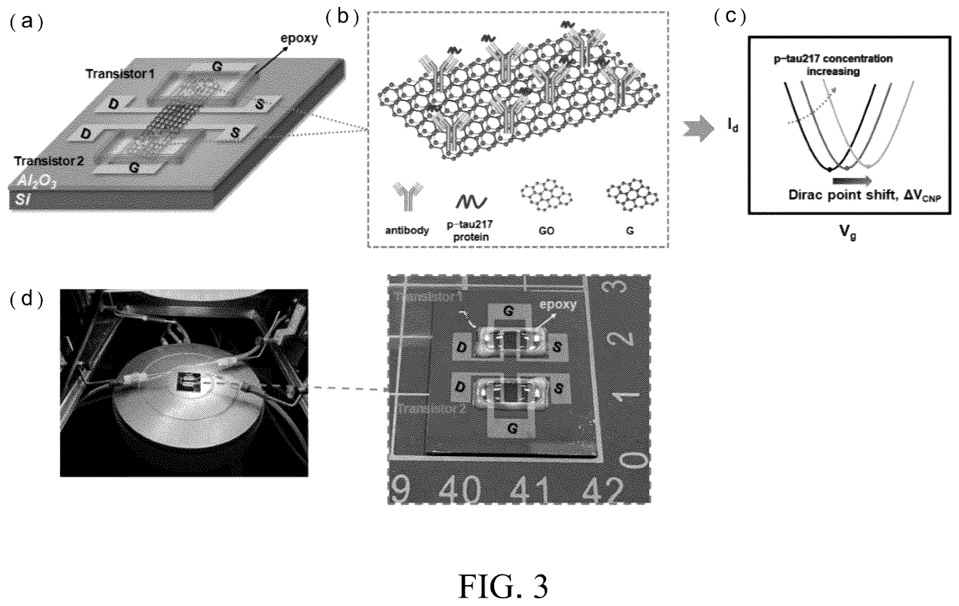

FIG. 1 shows the procedure of manufacturing the biosensor of the present disclosure. A 6 nm aluminum oxide (Al₂O₃) film was deposited onto a Si substrate using an atomic layer deposition system to obtain an Al₂O₃/Si substrate with size of 3×2.5 cm². Electrical contacts of Cr/Au (5/50 nm) were deposited onto the Al₂O₃/Si substrate to establish a source, drain and planar gate using a thermal evaporator through a shadow mask. Two sets of electric contacts were prepared on the substrate, and the detailed dimensions are displayed in FIG. 2. Then, the bilayer graphene (BG), formed by the chemical vapor deposition (CVD) process (Govindasamy et al., 2022), was transferred to the substrate across the source and drain electrodes followed by thermal annealing under a vacuum environment to remove any other polymer residues and enhance the contact between the BG and the electrodes to obtain BG on the Al₂O₃/Si substrate (BG/Al₂O₃/Si). Afterward, the bilayer graphene was subjected to LDPT with gas mixtures of oxygen and hydrogen to form the GO/G layered composite on the electrode-deposited Al₂O₃/Si substrate. The details of the LDPT process have been reported previously (Govindasamy et al., 2022). Finally, an epoxy resin-type adhesive was dispensed to define the sensing area of 4×9 mm² for each set of devices. Immediately after preparing the GO/G layered composite on the electrode-deposited Al₂O₃/Si substrate, the antibody was immobilized onto the GO/G surface via a carbodiimide-mediated reaction between the amine groups (—NH₂) of the aminated antibody at the C-terminus and the carboxyl groups (—COOH) of the GO. A 20 μl aliquot of 100 μg/ml antibody solution was dispensed onto the sensing region and incubated at 4° C. for 16 h. Next, the antibody solution-incubated devices were gently rinsed with PBS three times to wash away the excess nonbonded antibody. A total of 20 μl of p-tau217 solutions with various concentrations in PBS were added dropwise onto the sensing region at room temperature for 30 min to bind with the antibody. Then, the samples were gently rinsed with PBS to remove the excess unbound target p-tau217 proteins. Also, different concentrations of p-tau217 solution in HSA were prepared with the same protocol as that in PBS to verify the practicality of the detection method. FIG. 3 shows a schematic illustration of the SGFET-based biosensor featuring a GO/G layered composite for the p-tau217 protein.

### Characterization of the BG and GO/G Layered Composite

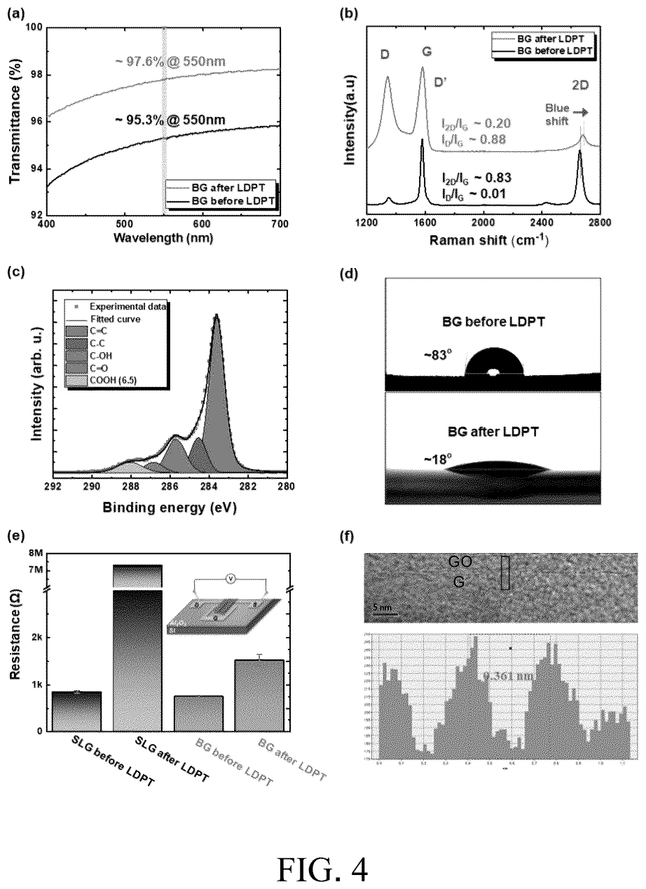

The GO/G layered composite was provided. The top layer of GO reacts with the antibody acting as the biorecognition element, and the bottom layer of G serves as a transducer. The preparation of the GO/G layered composite started with the growth of BG followed by LDPT. UV-Vis spectroscopy, Raman spectroscopy, XPS, CA measurements, and electrical resistance measurements were performed to examine the formation of the GO/G layered composite (FIG. 4). FIG. 4(a) shows the UV-Vis spectra of BG before and after LDPT. The transmittance of the BG before LDPT at 550 nm was 95.3%, which was almost in agreement with the transmittance reduction of 2.3% for single-layer graphene (SLG). The Raman spectra displayed in FIG. 4(b) show that the ratio of the intensities of 2D to G for the BG before LDPT is approximately 0.83 with low intensity in the D band, and the full width at half-maximum of the 2D band is −59 cm, suggesting the formation of AB-stacked BG (Sheng et al., 2015; Liu et al., 2012). Given these material analyses, high-quality BG was used in the present disclosure. The transmittance of the BG after LDPT slightly increased. It has been reported that the band gap of oxidized graphene increases due to oxygen incorporation into graphene (Jin et al., 2020). Therefore, the light absorption in the visible range decreases, leading to an increase in transmittance as shown in FIG. 4(a). After the BG sample was subjected to LDPT, the intensity of the D band increased, and the intensity ratio of the 2D- to G-bands (I_{2D}/I_{G}) dramatically decreased, resulting from the incorporation of the oxidative functional groups into the BG. It is noted that a blueshift was observed after LDPT, indicating a p-doping effect on the BG (Tang et al., 2010). FIG. 4(c) shows the XPS spectra of the BG sample after LDPT. Five kinds of bonding states for the carbon atoms could be deconvoluted: an sp² C═C bond at 284.5 eV, sp3 C—C bond at 285.5 eV, hydroxyl group (C—OH) at 286.6 eV, carbonyl group (C═O) at 288 eV, and carboxyl group (COOH) at 288.9 eV (Krishnamoorthy et al., 2013; Huang et al., 2021). Those oxygen functional groups resulted in a defect structure in BG corresponding to an increase in the D band and a decrease in I_{2D}/I_{G} in the Raman spectra, as shown in FIG. 4(b). The COOH groups, the main functional unit required for covalent bond formation between the amino group of the antibody and the top layer of GO, contributed up to 6.5% of all the carbon bonding states. The hydrophilicity of the BG samples was evaluated in terms of their WCAs. As shown in FIG. 4(d), the BG sample became hydrophilic by observing the CAs changing from 83° before LDPT to 18° after LDPT. The results confirmed that a hydrophilic surface resulted from the formation of oxidative functional groups, consistent with the XPS spectra. FIG. 4(e) demonstrates the electrical resistances of BG and single-layer graphene (SLG) samples before and after LDPT between the source and drain electrodes. The resistance of the SLG increased dramatically after LDPT and exceeded several megaohms, indicating that it almost became an electrically insulating material.

Conversely, the resistance of the BG after LDPT only slightly increased and maintained the same order of conductivity. Given these results, it was concluded that the top layer of G in the BG samples turned into GO, while the bottom layer of G was modified very little by the LDPT. TEM measurements were performed to confirm the layered G composite of GO/G. As shown in FIG. 4(f), a double-layer structure of GO/G was clearly observed, and the spacing between the two layers was approximately 0.361 nm. As per the results in FIG. 4, atomic layer oxidation was achieved through LDPT to form a layered G composite of GO/G as a transducer, while the top layer of GO could react with antibodies for subsequent antigen binding.

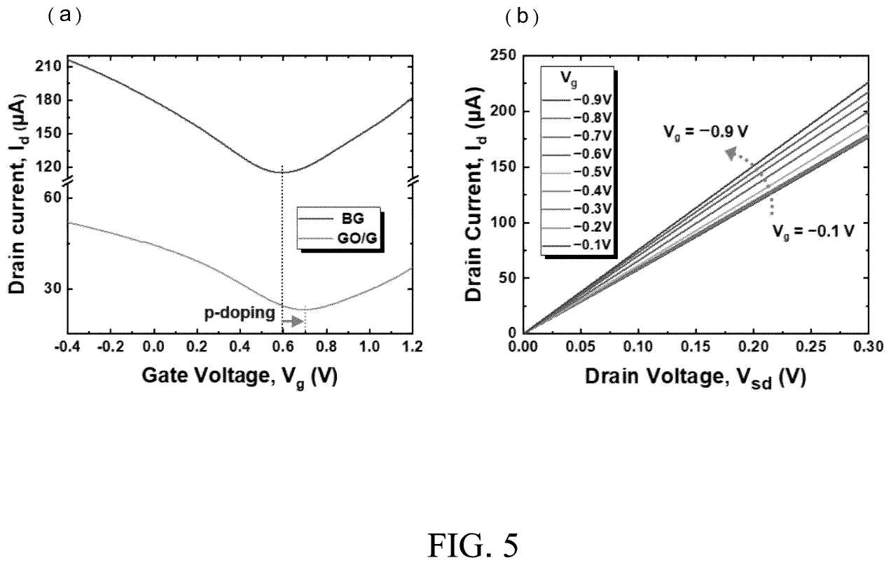

FIG. 5(a) plots the representative transfer curve (drain-source current (I_{d}) concerning gate voltage (V_{g})) of the SGFET featuring the BG. A typical feature of the V-shaped curve of the BG was observed, suggesting an ambipolar characteristic. The V_{CNP} was located at a positive V_{g} (~0.6 V), indicating p-type doping resulting from adsorbates during sample preparation, which has been widely observed for CVD-grown G (Pirkle et al., 2011; Liao et al., 2022). With LDPT, the BG sample was transformed into a GO/G layered composite, and the value of V_{CNP} of the SGFET shifted toward a more positive V_{g} because the presence of electronegative oxygen functionalities withdraws π-electrons from graphene, leading to its p-type behavior (Dey et al., 2016). FIG. 5(b) shows the electrical characteristics of the GO/G-based SGFET with the I_{d}—V_{sd} curves by changing V_{g} from −0.1 to −0.9 V. The value of I_{d} decreased significantly as the value of V_{g} slightly decreased, suggesting a sensitive modulation of electrical transport concerning the gate voltage.

Sensing Performance Toward p-Tau217

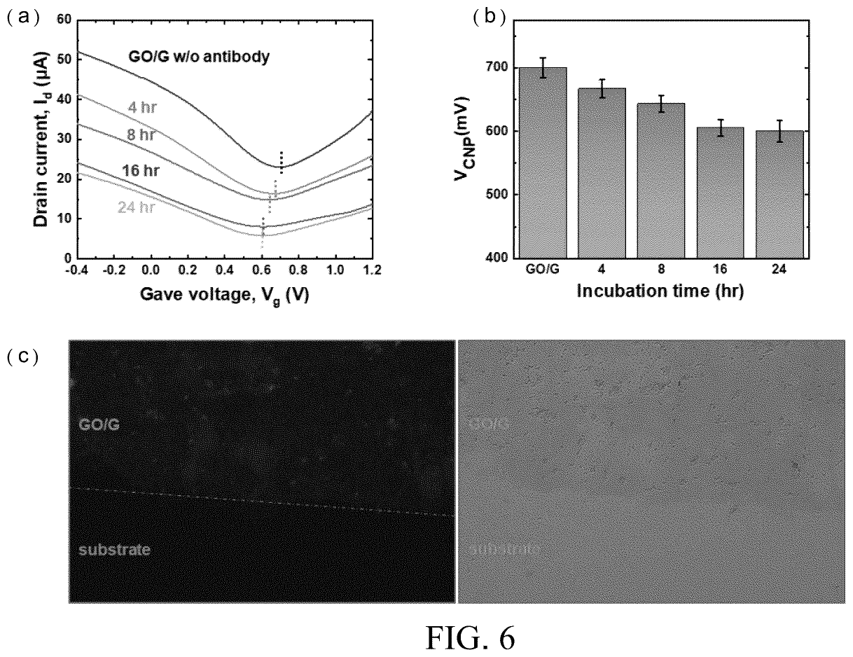

To optimize the sensing performance of the SGFET-based biosensor, the incubation time for antibody immobilization on the GO/G surface was investigated. The I_{d}—V_{g} transfer curves were measured after the antibody immobilization process, as shown in FIG. 6(a), and the VCNP values in FIG. 6(b) were extracted from each transfer curve. When the incubation time was 4 hr, the V_{CNP} shifted toward a negative V_{g}, indicating n-type doping. The negative charge of the antibody in the present disclosure is consistent with the literature. As the immobilization time increased to 16 hr, the V_{CNP} continued shifting toward a more negative V_{g}, indicating that more antibodies were immobilized on the GO/G surface. It is noted that the V_{CNP} was almost the same when the incubation time was extended to 24 hr, suggesting that the amount of immobilized antibody on the GO/G surface became saturated. Therefore, an incubation time of 16 hr was used for the subsequent p-tau217 detection. Also, the GO/G sample was exposed to an amine- and FAM (carboxy fluorescein)-modified antibody, and then antibody immobilization on the GO/G surface was examined with an incubation time of 16 hr. FIG. 6(c) shows fluorescence microscopy image of the GO/G sample after exposure to the amine- and FAM-modified antibody (left) and the corresponding optical image (right). The fluorescence observed on the GO/G, outlined by the dotted lines, confirmed the successful immobilization of the antibody through the incubation process.

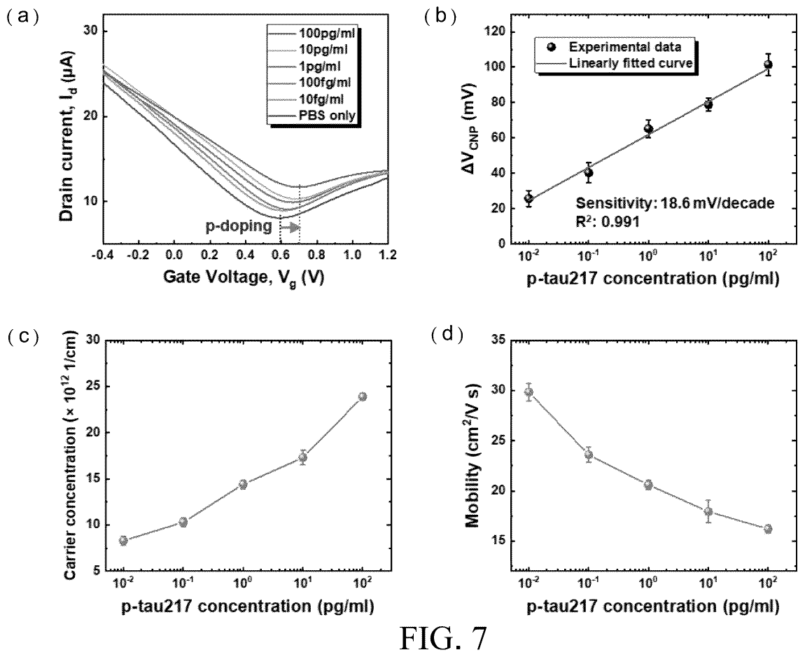

After optimization of the incubation time of the antibody, the sensing performance of the GO/G-based SGFET biosensors toward target p-tau217 proteins at various concentrations was determined. The detection of the target p-tau217 was performed with tenfold increases from 10 fg/ml to 100 μg/ml in sequence. As shown in FIG. 7(a), the V_{CNP} shifted toward the positive V_{g} upon the binding of the target p-tau217. It has been reported that p-tau217 has a high positive charge density surrounding the aggregation-prone microtubule-binding region and long-range N-terminal interactions with the mid-domain and C-terminal domain (Limorenko et al., 2022). Therefore, it was considered that this rightward shift upon binding of the target p-tau217 with the GO/G-based SGFET biosensor resulted from the charge transfer from the positively charged p-tau217 to the GO/G active channel imposing a p-type doping effect because the proximity of the target p-tau217 protein to the graphene composite surface led to Coulombic interactions between it and the hole-rich tau proteins. As the concentration of the target p-tau217 increased, a greater rightward shift occurred owing to more hole charge transfer coming from the target p-tau217 proteins. FIG. 7(b) is a graph showing the shift in the value of V_{CNP} after target binding relative to that before binding, denoted as ΔV_{CNP}, concerning the logarithmic concentration.

The biosensor of the present disclosure showed a high sensitivity of 18.6 mV/decade with linearity (R²) of 0.991 and a limit of detection (LOD) of 10 fg/ml. Since the present disclosure provides the first demonstration of p-tau217 detection using a nanomaterial-based POC biosensor, a comparison with other methods is not available. Previously reported LODs of the other p-tau isoform, p-tau231, are 4.71 pg/ml with a colorimetric gold nanoparticle-based aptablot (Phan et al., 2022) and 60 pg/ml with an electrochemical biosensor based on a wave-shaped gold thin film electrode (Le et al., 2022). These LODs are much higher than those in the present disclosure. Hall measurements were conducted to examine the p-type doping effect resulting from the binding of the target p-tau217 proteins using the same immobilization and binding protocol as that described previously in the present disclosure. As shown in FIG. 7(c), the carrier concentration (n) of the majority carriers of the holes in the GO/G increased as the concentration of the target p-tau217 proteins increased. The result confirmed hole transfer from the positively charged p-tau217 proteins to the GO/G responsible for the rightward shift of V_{CNP}. The mobility (φ of GO/G depending on the concentration of the target p-tau217 proteins was also obtained in Hall measurements (FIG. 7(d)). The mobility decreased upon increasing the concentration of the target p-tau217 proteins. It is well known that G is a typical 2D material possessing high sensitivity to the attachment of foreign atoms or molecules leading to carrier scattering within it. Therefore, more attachment of the target p-tau217 proteins leads to more scattering in the bottom layer of G through π-π interactions between GO and G and thus a lower carrier mobility. It is noted that the carrier concentration increased approximately threefold while the mobility decreased approximately twofold throughout the whole concentration variation range. Based on the van der Pauw equation:

$$R ∝ 1 / μ n$$

where R is the resistivity. R is proportional to the inverse of the product of μ and n. n causes an approximately threefold reduction of R, and μ causes an approximately twofold increase of R. Overall, the R slightly decreased. This may explain the slight increase in I_{d} upon increasing the amount of target p-tau217 proteins.

### Specificity and Stability of Biosensor

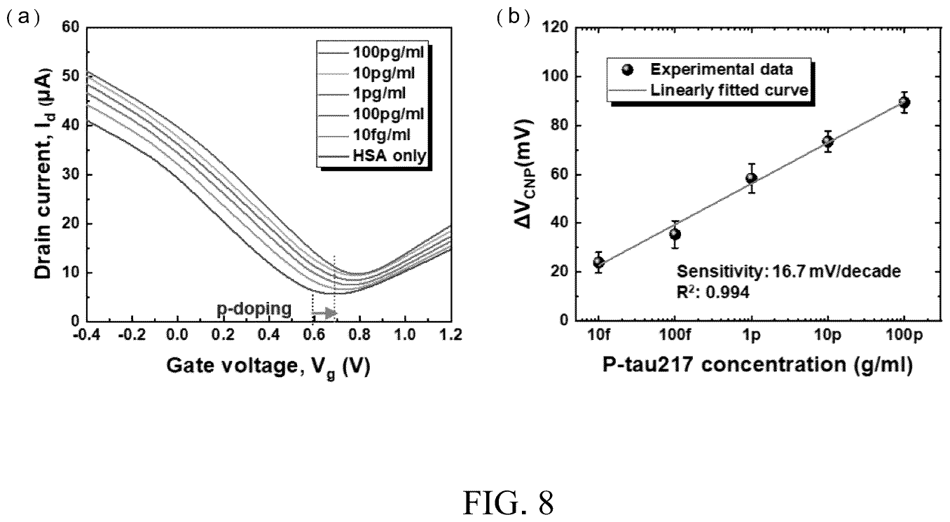

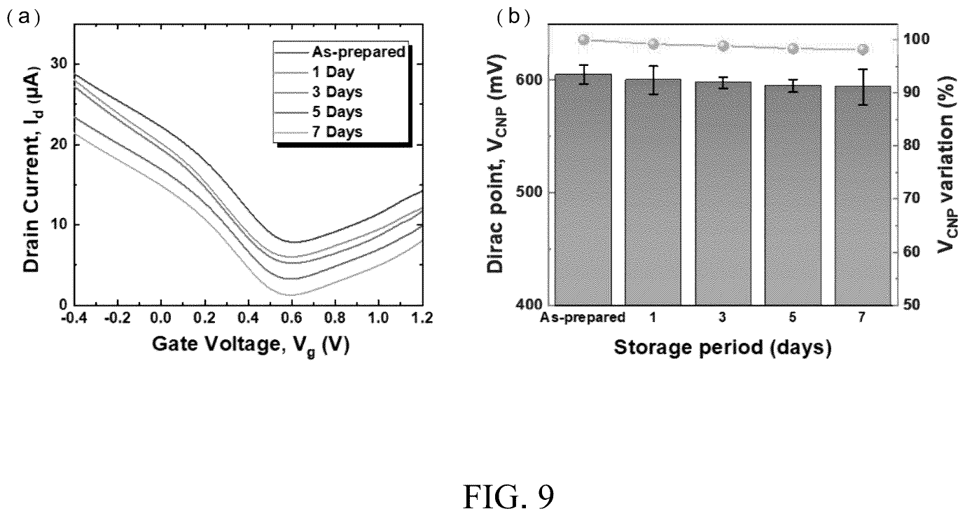

Specific sensing is in high demand for the clinical testing of any biosensor. In the present disclosure, the specificity of the GO/G-based SGFET biosensor toward p-tau217 proteins was examined in undiluted HSA at the same concentration range as that tested in PBS with the same detection protocol. FIG. 8(a) shows the representative transfer curves of the GO/G-based SGFET measured at various concentrations of the target p-tau217 proteins. Ambipolar transport behavior was also observed in HSA solution. The value of V_{CNP} also shifted toward positive V_{g} upon increasing the concentration of the p-tau217 proteins. The trend of the I_{d} change depending on the concentration of p-tau217 proteins was almost the same as that measured in PBS. FIG. 8(b) shows the plotted values of ΔV_{CNP} concerning V_{CNP} measured in only HSA solution. The sensitivity was 16.7 mV/decade based on linear fitting with a high value of R² (0.994). It is noted that the sensitivity still remained at approximately 90% of that measured in PBS, suggesting high specificity in a complex matrix. Stability is another crucial factor for assays in clinical testing. To examine the stability of the GO/G-based SGFET biosensor, the transfer curves of the antibody-modified GO/G-based SGFET after antibody immobilization, which were ready for target p-tau217 protein detection, were measured. Before each measurement, the antibody-modified GO/G-based SGFET were stored in a PBS solution. FIG. 9(a) shows the representative transfer curves of the antibody-modified GO/G-based SGFET obtained after varying days of storage. As shown in FIG. 9(b), the values of V_{CNP} were extracted from each of the curves. The values of V_{CNP} remained almost the same even after seven days of storage, with variation less than 2%. It is noted that I_{d} gradually and slightly decreased with the number of days in storage because the S and D electrodes were repeatedly contacted by the needle probes, leading to inevitable electrode deterioration. Although the I_{d} reduction resulted from the repeated contact, the intrinsic characteristic of the G layered composite as the sensing mechanism, i.e., the V_{CNP} shift was very stable, demonstrating the good stability of the biosensor manufactured in the present disclosure.

The present disclosure provides an SGFET-based biosensor featuring a GO/G layered composite to detect p-tau217 protein. This was the first study to detect the most efficacious biomarker of AD. The top layer of GO covalently immobilized the amine-functionalized antibody; and the bottom layer of G acted as a transducer to respond to the attachment of the target p-tau217 proteins via π-π interactions between GO and G layers. The value of ΔV_{CNP} of the biosensor increased linearly upon increasing the logarithmic concentration of the target p-tau217 protein (from 10 fg/ml to 100 pg/ml), with a sensitivity of 18/6 mV/decade, linearity of 0.991, and a LOD of 10 fg/ml in a PBS environment. Also, the detection in HSA was performed so as to obtain approximately 90% sensitivity in HSA with similar linearity and LOD. In addition to the excellent specificity, the stability of the antibody-coated biosensor, which is ready for sensing, was confirmed by observing only approximately 2% variation after storage in PBS for 7 days. Accordingly, it is believed that the SGFET-based biosensor manufactured by the method of the present disclosure advances the accuracy of early diagnosis of AD.

While the present disclosure has been described by means of specific embodiments, numerous modifications and variations could be made thereto by those skilled in the art without departing from the scope and spirit of the present disclosure set forth in the claims.

What is claimed is:

1. A method of manufacturing a biosensor for detecting a biomarker of Alzheimer's disease, comprising steps of:
depositing an aluminum oxide film on a Si substrate by an atomic layer deposition system to form an Al₂O₃/Si substrate, wherein a surface of the Si substrate is covered by the aluminum oxide film;
depositing electrical contacts Cr/Au on the Al₂O₃/Si substrate by a thermal evaporator to form a source, a drain and a planar gate on the Al₂O₃/Si substrate;
subjecting the bilayer graphene on the Al₂O₃/Si substrate across the source and the drain by thermal annealing under a vacuum environment;
providing a bilayer graphene to a low-damage plasma treatment (LDPT) with a mixture of oxygen and hydrogen to form a graphene oxide/graphene (GO/G) layered composite on the Al₂O₃/Si substrate; and
immobilizing an antibody on a surface of the GO/G layered composite through a reaction between amine groups of the antibody and carboxyl groups of GO of the GO/G layered composites, wherein the antibody is specific for p-tau217 protein.

2. The method according to claim 1, further comprising a step of dispensing an epoxy resin-type adhesive to define a sensing area.

3. The method according to claim 2, wherein the sensing area has a size in a range from 5×5 to 10×10 mm².

4. The method according to claim 2, wherein the step of immobilizing the antibody on the surface of the GO/G layered composite is performed by incubating the sensing area with a volume in a range from 20 to 100 μl aliquot of 100 μg/ml of the antibody at a temperature in a range from 4 to 37° C. for I to 24 hours.

5. A biosensor for detecting a biomarker of Alzheimer's disease, fabricated by a method comprising steps of:
depositing an aluminum oxide film on a Si substrate by an atomic layer deposition system to form an Al₂O₃/Si substrate, wherein a surface of the Si substrate is covered by the aluminum oxide film;
depositing electrical contacts Cr/Au on the Al₂O₃/Si substrate by a thermal evaporator to form a source, a drain and a planar gate on the Al₂O₃/Si substrate;
providing a bilayer graphene on the Al₂O₃/Si substrate across the source and the drain by thermal annealing under a vacuum environment;
subjecting the bilayer graphene to a low-damage plasma treatment (LDPT) with a mixture of oxygen and hydrogen to form a graphene oxide/graphene (GO/G) layered composite on the Al₂O₃/Si substrate; and
immobilizing an antibody on a surface of the GO/G layered composite through a reaction between amine groups of the antibody and carboxyl groups of GO of the GO/G layered composites, wherein the antibody is specific for p-tau217 protein.

6. A biosensor for detecting a biomarker of Alzheimer's disease, comprising:
a Si substrate;
an aluminum oxide film disposed on the Si substrate by an atomic layer deposition system to form an Al₂O₃/Si substrate, wherein a surface of the Si substrate is covered by the aluminum oxide film;
electrical contacts Cr/Au disposed on the Al₂O₃/Si substrate by a thermal evaporator;
a graphene oxide/graphene (GO/G) layered composite formed on the Al₂O₃/Si substrate by a low-damage plasma treatment (LDPT); and
an antibody immobilized on a surface of the GO/G layered composite through a reaction between amine groups of the antibody and carboxyl groups of GO of the GO/G layered composites, wherein the antibody is specific for p-tau217 protein.

7. The biosensor according to claim 6, wherein an epoxy resin-type adhesive is dispensed to define a sensing area.

8. The biosensor according to claim 7, wherein the sensing area has a size in a range from 5×5 to 10×10 mm².

9. The biosensor according to claim 7, wherein the antibody is immobilized on the surface of the GO/G layered composite by incubating the sensing area with a volume in a range from 20 to 100 μl aliquot of 100 μg/ml of the antibody at a temperature in a range from 4 to 37° C. for 1 to 24 hours.

10. A method for detecting a biomarker of Alzheimer's disease, comprising a step of:
detecting an amount of p-tau217 protein in a sample with a biosensor fabricated by steps of:
depositing an aluminum oxide film on a Si substrate by an atomic layer deposition system to form an Al₂O₃/Si substrate, wherein a surface of the Si substrate is covered by the aluminum oxide film;
depositing electrical contacts Cr/Au on the Al₂O₃/Si substrate by a thermal evaporator to form a source, a drain and a planar gate on the Al₂O₃/Si substrate;
subjecting the bilayer graphene on the Al₂O₃/Si substrate across the source and the drain by thermal annealing under a vacuum environment;
providing a bilayer graphene to a low-damage plasma treatment (LDPT) with a mixture of oxygen and hydrogen to form a graphene oxide/graphene (GO/G) layered composite on the Al₂O₃/Si substrate; and
immobilizing an antibody on a surface of the GO/G layered composite through a reaction between amine groups of the antibody and carboxyl groups of GO of the GO/G layered composites, wherein the antibody is specific for the p-tau217 protein.

11. The method according to claim 10, wherein an epoxy resin-type adhesive is dispensed to define a sensing area of the biosensor.

12. The method according to claim 11, wherein the sensing area has a size in a range from 20 to 80 mm².

13. The method according to claim 11, wherein the antibody is immobilized on the surface of the GO/G layered composite by incubating the sensing area with a volume in a range from 20 to 100 μl aliquot of 100 μg/ml of the antibody at a temperature in a range from 4 to 37° C. for 1 to 24 hours.

14. A method for detecting a biomarker of Alzheimer's disease, comprising a step of:
detecting an amount of p-tau217 protein in a sample with a biosensor, wherein the biosensor comprises:
a Si substrate;
an aluminum oxide film disposed on the Si substrate by an atomic layer deposition system to form an Al₂O₃/Si substrate, wherein a surface of the Si substrate is covered by the aluminum oxide film;
electrical contacts Cr/Au disposed on the Al₂O₃/Si substrate by a thermal evaporator;
a graphene oxide/graphene (GO/G) layered composite formed on the Al₂O₃/Si substrate by a low-damage plasma treatment (LDPT); and
an antibody immobilized on a surface of the GO/G layered composite through a reaction between amine groups of the antibody and carboxyl groups of GO of the GO/G layered composites, wherein the antibody is specific for the p-tau217 protein.

15. The method according to claim 14, wherein an epoxy resin-type adhesive is dispensed to define a sensing area of the biosensor.

16. The method according to claim 15, wherein the sensing area has a size in a range from 5×5 to 10×10 mm².

17. The method according to claim 15, wherein the antibody is immobilized on the surface of the GO/G layered composite by incubating the sensing area with a volume in a range from 20 to 100 μl aliquot of 100 μg/ml of the antibody at a temperature in a range from 4 to 37° C. for 1 to 24 hours.
